(19) United States
(12) Patent Application Publication
(10) Pub. No.: US 2026/0363616 A1
(43) Pub. Date: Nov. 19, 2026

(54) ESTIMATING SLEEP GATE

(71) Applicant: Polar Electro Oy, Kempele (FI)

(72) Inventors: Topi KORHONEN, Kempele (FI); Riikka AHOLA, Kempele (FI); Matti Luomala, Kempele (FI); Kaisu Martinmäki, Kempele (FI); Lotta Rönnberg, Kempele (FI); Eve Vallström, Kempele (FI)

(73) Assignee: Polar Electro Oy, Kempele (FI)

(21) Appl. No.: 19/126,258

(22) PCT Filed: Nov. 2, 2023
(86) PCT No.: PCT/FI2023/050612

§ 371 Date: May 1, 2025

(30) Foreign Application Priority Data
Nov. 4, 2022 (GB) 2216411.5

(51) Int. Cl. A61B 5/00; A61B 5/01

(52) U.S. Cl. CPC A61B 5/4857; A61B 5/01; A61B 5/4806; A61B 5/681; A61B 5/742

(57) ABSTRACT

A computer-implemented method for estimating sleep gate of a user includes: obtaining measurement data measured, from a user, by at least one sensor device; determining, using the measurement data obtained, a circadian rhythm of the user, wherein the circadian rhythm comprises a circadian phase; determining, using the measurement data obtained, a sleep-wake rhythm of the user; determining, using the circadian phase determined, a sleep gate, wherein the sleep gate indicates a time window for falling asleep suitable for the circadian rhythm of the user; determining, using the sleep-wake rhythm determined, a reliability metric value indicating a timing accuracy of the sleep gate; and causing displaying, to the user via a user interface, the sleep gate and the reliability metric value.

### CROSS-REFERENCE TO RELATED APPLICATIONS

This application is a national phase application of International Application No. PCT/FI 2023/050612, filed Nov. 2, 2023, which claims benefit and priority to Great Britain Application No. 2216411.5, filed Nov. 4, 2022, which are incorporated by reference herein in their entireties.

### BACKGROUND

### Technical Field

The invention relates to a field of measuring a human and, in particular, to estimating an ideal sleep gate using measurements.

### Summary

Modern activity monitoring devices, sometimes called activity trackers, employ motion sensors to measure user's motion during the day. Some activity monitoring devices may employ other sensors such as physiological or biometric sensors such as heart activity sensors. Some activity monitoring devices are also capable of estimating sleep time and/or sleep quality of the user, which may be used for estimating and improving timing of sleep.

In a human, melatonin is secreted at night in accordance with a circadian rhythm of the human. The rise of melatonin secretion correlates with a subsequent increase in sleep propensity about two hours before the human's regular bedtime. The time before this secretion is the least likely for sleep to occur, and when it starts, the propensity for sleep increases greatly as this “sleep gate” opens. The rhythmic release of melatonin is regulated by the central circadian rhythm generator of the human body, the suprachiasmatic nucleus (SCN) of the anterior hypothalamus. The sleep gate thus refers to a steep rise in sleepiness that marks the start of a period characterised by consistently high levels of the sleep propensity. It is possible to estimate the sleep gate by measuring the circadian rhythm, but improvements are needed.

According to an aspect, there is provided the subject matter of the independent claims. Embodiments are defined in the dependent claims.

### BRIEF DESCRIPTION OF THE DRAWINGS

In the following, various example embodiments will be described in greater detail with reference to the accompanying drawings, in which
FIG. 1 illustrates a simplified architecture of a system;

FIGS. 2 to 4 are flow charts illustrating example functionalities;

FIGS. 5a and 5b illustrate display views according to some embodiments; and
FIG. 6 illustrates an exemplary embodiment of an apparatus.

### DETAILED DESCRIPTION

The following embodiments are exemplary. Although the specification may refer to “an”, “one”, or “some” embodiment(s) in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments/examples to consist of only those features that have been mentioned and such embodiments may contain also features/structures that have not been specifically mentioned. Further, although terms including ordinal numbers, such as “first”, “second”, etc., may be used for describing various elements, the structural elements are not restricted by the terms. The terms are used merely for the purpose of distinguishing an element from other elements. For example, a first element could be termed a second element, and similarly, a second element could be also termed a first element without departing from the scope of the present disclosure.

Different embodiments and examples are described below using single units, models, equipment, and memory, without restricting the embodiments/examples to such a solution. Concepts called cloud computing and/or virtualization may be used. The virtualization may allow a single physical computing device to host one or more instances of virtual machines that appear and operate as independent computing devices, so that a single physical computing device can create, maintain, delete, or otherwise manage virtual machines in a dynamic manner. It is also possible that device operations will be distributed among a plurality of servers, nodes, devices, or hosts. In cloud computing network devices, computing devices and/or storage devices provide shared resources. Some other technology advancements, such as Software-Defined Networking (SDN), may cause one or more of the functionalities described below to be migrated to any corresponding abstraction or apparatus or device. Correspondingly, Web 3.0, also known as the third-generation internet, implementing for example blockchain technology, may cause one or more of the functionalities described below to be distributed across a plurality of apparatuses or devices. Therefore, all words and expressions should be interpreted broadly, and they are intended to illustrate, not to restrict, the embodiment.

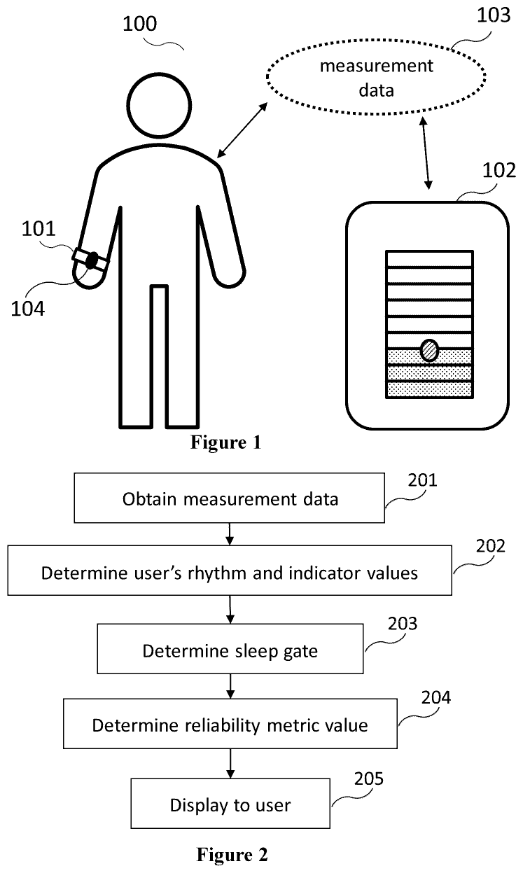

A general exemplary architecture of a system estimating a sleep gate of a user is illustrated in FIG. 1. FIG. 1 is a simplified system architecture showing only some devices, apparatuses, and functional entities, all being logical units whose implementation and/or number may differ from what is shown. The connections shown in FIG. 1 are logical connections; the actual physical connections may be different. It is apparent to a person skilled in the art that the system comprises any number of shown elements, other equipment, other functions, and other structures that are not illustrated. They, as well as the protocols used, are well known by persons skilled in the art and are irrelevant to the actual invention. Therefore, they need not to be discussed in more detail here.

In the example illustrated in FIG. 1, the system 100 comprises at least a processing circuitry configured to analyse measurement data 103 measured from a user, for example by carrying out methods described in more detail below. The processing circuitry may be realised in a wearable device 101 worn by the user, such as a smart watch or wrist-worn wearable training computer or activity tracker device. The processing circuitry may be realised in a user device 102 such as a smart phone or a tablet computer. The processing circuitry may be realised in a server computer such as a cloud server. The measurement data 103 may be provided by at least one sensor device 104 which may be comprised in the wearable device 101, or the sensor device may be external to the wearable device 101 but provided with data transfer capability with the wearable device 101. The wearable device 101, the user device 102, the server computer, and/or the sensor device 104 may be connectable over one or more networks, over a short-range wireless connection such as Bluetooth, or over a Universal Serial Bus (USB) connection.

The sensor device 104 may measure one or more of the following features from the user: motion, electrocardiogram (ECG), photoplethysmogram (PPG), electroencephalography (EEG), bioimpedance, galvanic skin response, body temperature, respiration, electrooculogprahy (EOG), or ballistocardiogram (BCG). For measuring motion, the sensor device 104 may comprise an inertial sensor such as an accelerometer and/or gyroscope, or a magnetometer, or any a sensor fusion that is any combination of these motion sensors, and the sensor device 104 may output motion measurement data. The sensor device 104 measuring ECG, PPG, or BCG may output heart activity measurement data 103. The sensor device 104 may comprise one or more electrodes attachable to the user's skin to measure an electric property from the skin which, through appropriate signal processing techniques, may be processed into an ECG signal. In some techniques, the heart activity measurement data 103 may represent appearance of R waves of electric heart impulses. In PPG measurements a light emitted by a light emitter diode or a similar light source and reflected back from the user's skin is be sensed by using a photo diode or a similar light sensing component. The sensed light is then converted into an electric measurement signal in the light sensing component and signal processing is used to detect desired signal components from the electric measurement signal. In the PPG measurements, P waves may be detected which enables computation of a PP interval and a heart rate, for example. The sensor device 104 measuring the EOG may output electric measurement data 103 representing eye motion. The sensor device 104 may comprise a special-purpose respiration sensor outputting a respiratory rate. The respiratory rate may also be measured from the heart activity measurements.

The user device 102 refers to a computing device (equipment, apparatus) and it may also be referred to as a user terminal, a user apparatus, mobile device, or a mobile terminal. Portable computing devices (apparatuses) include wireless mobile communication devices operating with or without a subscriber identification module (SIM) in hardware or in software, including, but not limited to, the following types of devices: mobile phone, smartphone, personal digital assistant (PDA), handset, laptop and/or touch screen computer, tablet (tablet computer), multimedia device, wearable computer, such as smart watch, and other types of wearable devices, such as clothing and accessories incorporating computer and advanced electronic technologies. The user device 102 may comprise one or more user interfaces. The one or more user interfaces may be any kind of a user interface, for example a screen, a keypad, a loudspeaker, a microphone, a touch user interface, an integrated display device, and/or external display device.

According to the example illustrated in FIG. 2 a sleep gate and a reliability metric value are estimated for a user, for example by the wearable device 101, the user device 102, or the server computer.

The term “sleep gate” can be understood as a short time period when a user (person) is falling asleep. Due to normal life practices, falling asleep does not happen immediately when you want, or when some instruction is guiding you to do so. Thus, “the sleep gate” describes better the time window (frame) when the user is preparing, approaching and finally falling asleep. It can be understood as a time window around the go-to-sleep time moment or event, or time immediately starting from the go-to-sleep time. In one embodiment, it is possible to calculate the sleep gate time as a time point indicating an optimal sleep start point, and then define a time window around the time point. For example, one hour before and one hour after the time point forms the sleep gate period of two hours, or 30 minutes before and after the time point forms the sleep gate period of one hour, or 15 minutes before and after the time point forms the sleep gate period of 30 minutes. The period of two hours or one hour or 30 minutes can be also started from the time point. It is also possible to define any other time period less than two hours before and/or after the time point. The time point may locate anywhere within the defined time period, then time before the time point may also be shorter or longer than time after.

Referring to FIG. 2, measurement data measured from the user by at least one sensor device is obtained in block 201. The measurement data obtained may comprise various measurement data types as explained earlier with FIG. 1. The measurement data may be obtained from a memory or a data storage of the sensor device, the wearable device, the user device, and/or the server computer. A rhythm of the user is determined in block 202 from the measurement data obtained. The rhythm determined comprises a circadian rhythm of the user. The circadian rhythm (equivalently circadian process, circadian cycle) may be understood as an internal physiological process regulating a natural sleep-wake rhythm of the user. The circadian rhythm repeats approximately every 24 hours. The circadian rhythm period may vary person-by-person around the 24 hours. The circadian cycle comprises a circadian phase of the user. The circadian phase can be understood as reflecting timing of different states of the circadian rhythm during a (calendar) day.

The rhythm determined in block 202 also comprises a sleep-wake rhythm of the user. If the user follows the natural sleep-wake rhythm regulated by the suprachiasmatic nucleus (SCN) of the anterior hypothalamus (the circadian system), the sleep-wake rhythm would substantially follow the circadian rhythm. However, people often deviate from the natural sleep-wake rhythm because of social activities, personal preferences to stay awake during weekends, travelling across time zones, shift work, etc. As a consequence, the sleep-wake rhythm of the user may either follow the circadian rhythm or deviate from it, and this affects also the accuracy of the sleep gate, as described in greater detail below. This results from the fact that the sleep-wake rhythm affects the sleep propensity during the sleep gate via sleep homeostasis known in the literature. Excessive sleep reduces the sleep propensity during the sleep gate and misalignment between the sleep-wake rhythm and the circadian rhythm may shift the optimal bedtime from the timing of the sleep gate. The sleep-wake rhythm follows the user's actions of going to sleep and waking up, while the circadian rhythm changes at a slower pace. On the other hand, shortened primary sleep causes sleep debt and extra sleepiness before the sleep gate. Primary sleep refers to the sleep that the user takes daily and that is usually 6 to 10 hours long, depending on the person.

The sleep-wake rhythm may be measured by using state-of-the-art sleep analysis sensors and metrics. There exists a vast amount of literature and commercially available products for measuring the sleep start and sleep end times that define the sleep-wake rhythm and, therefore, detailed description thereof is omitted. For example, the sleep analysis functions used in Polar Vantage V or Polar Grit X may be employed to determine the sleep-wake analysis. The sleep start time and the sleep end time may be measured by using the above-described heart activity sensor and/or a motion sensor.

Additionally, one or more indicator values related to the user's sleep-wake rhythm and/or the circadian rhythm may be determined in block 202.

Referring to FIG. 2, a sleep gate is determined in block 203 using the circadian phase determined. The sleep gate indicates a time window for falling asleep suitable for the circadian rhythm of the user, that is, the sleep gate anticipates a time period when the user's body may be ready to fall asleep, as described in Background above. The sleep gate may be understood as a time interval starting a circadian sleep window. The circadian sleep window may be understood as a time period for a primary sleep event in the circadian rhythm of the user. The circadian sleep window may be understood as starting from a midpoint of the sleep gate and ending at a circadian wake-up time. A reliability metric value for the sleep gate is determined in block 204 using the sleep-wake rhythm determined. The reliability metric value indicates a timing accuracy of the sleep gate. Displaying to the user the sleep gate and the reliability metric value is caused in block 205 via a user interface.

The display of the sleep gate indicates to the user the optimal bedtime and, thus, guides the user to a healthier sleep and to improved overall health. As known in the art of life sciences, a regular daily rhythm has great benefits to alertness and health in general. The display of the reliability metric indicates to the user the accuracy of the sleep gate and further improves the guidance by informing the user when to follow the sleep gate guidance and when to follow other indicators (e.g. the personal feelings of sleepiness).

The measured sleep-wake rhythm and the circadian rhythm may be measured at the same time, e.g. for the same time interval of one or more 24-hour cycles. Both measurements may be carried out daily so that the sleep gate and the associated reliability metric value are indicated to the user on a daily basis. Blocks 203 and 204 may be computed in response to detecting that the user has woken up in the morning. This may trigger detection of the sleep end time and related computation of the sleep-wake rhythm for the coming day.

In an exemplary embodiment, the measurement data obtained comprises body temperature data of the user. The circadian rhythm is determined using the body temperature data, and the sleep gate is determined using the circadian rhythm and the body temperature data. As known in the art, the human circadian system modulates human metabolic heat production and, as a consequence, generates a rhythm to body temperature that follows the circadian rhythm. Both core body temperature and skin temperature of the human body follow this rhythm and, thus, by measuring either temperature it is possible to measure and estimate the circadian rhythm.

In an exemplary embodiment the one or more indicator values indicate a deviation between the circadian rhythm of the user and the sleep-wake rhythm of the user. The reliability metric value is determined using the deviation between the circadian rhythm and the sleep-wake rhythm such that a greater deviation results in a reliability metric value indicating a poorer reliability, that is, a poorer timing accuracy of the sleep gate. As described above, the misalignment of the sleep-wake rhythm and the circadian rhythm may change the optimal bedtime even outside the sleep gate based on the circadian rhythm. Therefore, the deviation indicating the misalignment triggers the indication of the sleep gate with lower reliability. This may indicate to the user that the user may give a lower weight to the bedtime recommendation of the sleep gate. On the other hand, if the deviation between the circadian rhythm and the sleep-wake rhythm is small and the resulting reliability metric value indicates high reliability of the sleep gate, the user is guided to give high weight to the recommendation of the sleep gate and to go to sleep during the sleep gate.

In an exemplary embodiment the determining the reliability metric value comprises selecting the reliability metric value amongst at least a first reliability metric value and a second reliability metric value, wherein the first reliability metric value indicates a higher reliability, that is, a higher timing accuracy of the sleep gate than the second reliability metric value. Provision of multiple reliability metric values enables indication of the accuracy of the sleep gate in greater detail. The reliability metric value may be provided in the form of a score value within a determined range, e.g. 1 to 10, 0 to 10, or 0 to 100. The reliability metric value may be provided in a verbal form, e.g. “High”, “Compromised”, and “Poor”. The reliability metric value may be provided in a coloured form of the sleep gate. For example, the sleep gate may be indicated in the form of a time interval (numeral or graphic), and the colour of the time interval may indicate the reliability, e.g. green for high reliability, orange or yellow for compromised reliability, and red for poor reliability.

A sleep event may also be a secondary sleep event. The secondary sleep refers to naps taken between the primary sleep events, and a nap lasts typically from 10 minutes to a couple of hours. The user typically enters various sleep stages including deep sleep and REM sleep during the primary sleep, in multiple sleep cycles, whereas the user conventionally does not enter the deep sleep stage during the secondary sleep, unless the nap is exceptionally long and the user very tired. One factor affecting the accuracy of the sleep gate is whether or not the user has taken a nap (secondary sleep) during the daytime, when during the daytime the nap has been taken, and the length of the nap. If the nap has not been taken, or if the nap occurred more than a determined time interval threshold (e.g. more than 6 hours) before the determined beginning of the sleep gate or nap has been short (less than the sleep time threshold, e.g. less than 30 minutes), the sleep gate accuracy may be considered high and the nap does not push the natural sleep time beyond the sleep gate. On the other hand, if the nap has been long, e.g. one or two hours, or it has occurred only a short time interval before the sleep gate, e.g. one or two hours, there is a great potential that the user's sleep homeostasis has changed so that the sleep propensity during the sleep gate is lower than that indicated by the circadian rhythm. As a consequence, the sleep gate may be considered to have poor reliability.

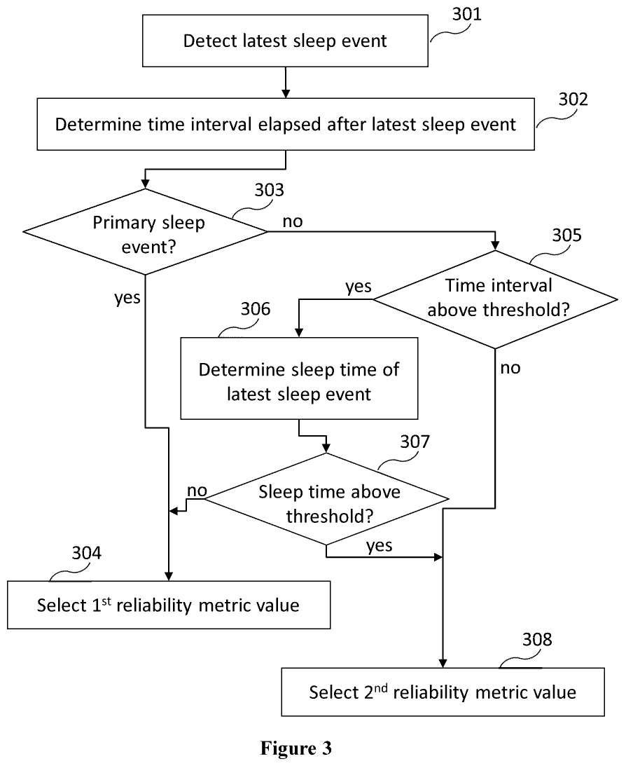

FIG. 3 illustrates an exemplary embodiment to determine the reliability metric value using a time interval elapsed after a secondary sleep event of the user and also using the sleep time of the secondary sleep event. The functionalities illustrated in FIG. 3 may be carried out within block 204 of FIG. 2. The time interval elapsed after the sleep event of the user affects the homeostatic sleep process of the user. As described above, the homeostatic sleep process may be understood as an amount of pressure for sleep, that is, tiredness, that builds up in a body of the user as awake-time increases. The build-up is subject to the user's sleep-wake rhythm. Referring to FIG. 3, a latest sleep event of the user is detected in block 301 from the measurement data obtained. The sleep event may be detected from the measurement data used for measuring the sleep-wake rhythm, e.g. the heart activity and/or motion measurement data. A time interval elapsed after the latest sleep event is determined in block 302. The time interval elapsed may comprise a time interval from (the end of) the latest sleep event to (the beginning of) the sleep gate. It is determined in block 303 whether or not the latest sleep event is a primary sleep event. If the latest sleep event is the primary sleep event (block 303: yes), the first reliability metric value indicating high reliability is selected in block 304 as the reliability metric value. If the latest sleep event is a secondary sleep event (a nap) and thus, not the primary sleep event (block 303: no), it is determined in block 305 whether the time interval elapsed is above a pre-determined time interval threshold. The time interval threshold may be pre-determined using, for example, the circadian rhythm of the user or the sleep-wake rhythm of the user, or it may be a pre-determined fixed value. If the time interval elapsed is above the time interval threshold (block 305: yes), a sleep time of the latest sleep event that is a secondary sleep event is determined in block 306. It is determined in block 307 whether the sleep time of the latest sleep event is above a pre-determined sleep time threshold. The sleep time threshold may be pre-determined using, for example, the circadian rhythm of the user or the sleep-wake rhythm of the user, or it may be a pre-determined fixed value. If the sleep time is above the sleep time threshold (block 307: yes), the second reliability metric indicating low reliability is selected in block 308 as the reliability metric value. If the sleep time is below the sleep time threshold (block 307: no), the first reliability metric value indicating high reliability is selected in block 304 as the reliability metric value. If the time interval elapsed is below the time interval threshold (block 305: no), the second reliability metric value indicating low reliability is selected in block 308 as the reliability metric value.

In an exemplary embodiment the one or more indicator values comprise a sleep debt value that is used in the determining the reliability metric value, for example, according to the principles disclosed in FIG. 3. As described above in connection with the sleep homeostasis, the sleep debt indicates that the user has had sleep deficiency during one or more previous primary sleep events, and the sleep debt may cause the natural sleep time to be earlier than the sleep gate. The sleep debt value may be estimated using the sleep-wake rhythm determined and a target sleep time preset for the user. The target sleep time may be preset by the user as a user input, or it may be determined using information received as a user input, such as, for example, age or activity, and be preset for the user. The target sleep time may also be determined using the sleep-wake rhythm determined over a longer period of time, e.g. over one or more weeks. The determining the reliability metric value comprises selecting the first reliability metric value indicating high reliability as the reliability metric value if the sleep debt value is below a sleep debt threshold, and selecting the second reliability metric value indicating poor reliability as the reliability metric value if the sleep debt value is above the sleep debt threshold. The sleep debt threshold may be pre-determined using, for example, the circadian rhythm of the user or the sleep-wake rhythm of the user, or it may be a pre-determined fixed value.

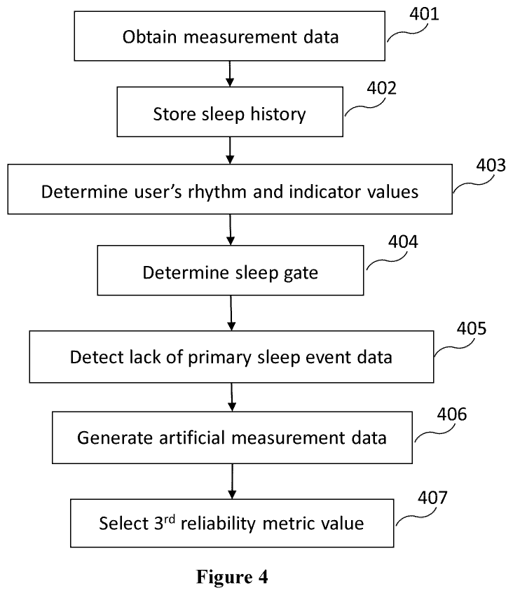

FIG. 4 illustrates an exemplary embodiment to determine the reliability metric value. According to the exemplary embodiment illustrated in FIG. 4 it is assumed that there is at least one gap in the measurement data, resulting in that artificial measurement data is generated. The gap may be caused by the user forgetting to wear the sensor device 104 during the primary sleep or a poor contact between the sensor device 104 and the user's skin. If the circadian rhythm measurements are based on the skin temperature data, it is possible that the skin contact has been poor during the primary sleep.

Referring to FIG. 4, measurement data measured from the user by the at least one sensor device 104 during at least one or more primary sleep events of the user is obtained in block 401. The measurement data obtained may comprise various measurement data types as explained above with FIG. 1. The measurement data may be obtained as described above with block 301 in FIG. 3. Sleep history data of the user is stored in block 402 in a memory. Naturally, the sleep history data may have been stored in the memory 402 as it has been recorded, so block 402 may be understood as maintaining the sleep history data in the memory. The sleep history data is based on the measurement data measured during the one or more earlier primary sleep events of the user. The sleep history data is determined using the measurement data obtained, for example by separating the measurement data measured during the one or more earlier primary sleep events of the user from other measurement data measured from the user, e.g. from motion data and/or heart activity data measured between a detected sleep end time and a following sleep start time (when the user has been awake). A rhythm of the user is determined in block 403 from the measurement data obtained. Also, one or more indicator values related to the user's sleep and/or rhythm may be determined in block 403. Some examples of indicator values are described above with FIG. 2. The sleep gate is determined in block 404 as described in more detail above with FIG. 2. A lack of measurement data indicating a primary sleep event of the user within a preceding 24-hour time period is detected in block 405. In response to the said detecting, artificial measurement data of an artificial primary sleep event is generated in block 406 using the sleep history data stored in the memory. A third reliability metric value is selected in block 407 as the reliability metric value. The third reliability metric value indicates a lower timing accuracy of the sleep gate than the first reliability metric value. The third reliability metric value may indicate even a lower timing accuracy than the second reliability metric value, or the third reliability metric value may indicate a greater timing accuracy than the second reliability metric value. In case the user's sleep-wake rhythm is relatively regular, the sleep history data may correlate with the user's sleep during the primary sleep for which no measurement data is available. However, even if the user has regular sleeping habits, one primary sleep event may still deviate substantially from another primary sleep event. Therefore, it is beneficial to indicate degraded reliability whenever the artificial measurement data is used for the sleep gate estimation.

In an exemplary embodiment, the artificial measurement data is a copy of sleep measurement data from one of the primary sleep events stored in the memory. The selected primary sleep event may have occurred on the same weekday as the day for which the measurement data is now missing, but from the previous week. This may improve the accuracy of the sleep gate because users often repeat the same weekly pattern.

In an exemplary embodiment, if the circadian rhythm has not been determined, for example, due to a lack of measurement data that may be caused by the user forgetting to wear the sensor device 104 during the primary sleep or a poor contact between the sensor device 104 and the user's skin, the sleep gate of a preceding day is determined as the sleep gate. However, even if the user has regular sleeping habits, one sleep gate may still deviate to some extent from another sleep gate. Therefore, it is beneficial to indicate degraded reliability whenever a preceding sleep gate is used for the sleep gate estimation. Hence, a fourth reliability metric value is selected as the reliability metric value. The fourth reliability metric value indicates a lower timing accuracy of the sleep gate than the first reliability metric value. The fourth reliability metric value may indicate even a lower timing accuracy than the second reliability metric value and/or the third reliability metric value, or the fourth reliability metric value may indicate a greater timing accuracy than the second reliability metric value and/or the third reliability metric value. Alternatively, the fourth reliability metric value may be equal to the third reliability metric value.

In an exemplary embodiment, the apparatus carrying out the procedure of FIG. 2 or any one of its embodiments stores, in the memory, a maximum limit defining how much at most a timing of the sleep gate can be changed at a time. Upon determining on the basis of the newly-calculated circadian phase that the circadian phase has shifted, the apparatus may determine to shift the sleep gate as well. As a consequence, the apparatus may compute a new timing for the sleep gate. Upon triggering a change in the timing of the sleep gate, the apparatus may use the maximum limit to limit the change in the timing. For example, if the maximum limit is two hours but the circadian phase indicates that the sleep gate should be shifted by three hours, the apparatus may limit the shift to two hours. In general, if the new timing causes a shift to the sleep gate that exceeds the maximum limit, the shift of the new timing is limited to the maximum limit, and the new timing is computed such that it complies with the maximum limit. In an embodiment, the maximum limit is different for clockwise shifting of the sleep gate than for counter-clockwise shifting of the sleep gate. This may account for the different adaptation of the user for “eastward” travel than for “westward” travel. It has been discovered that the user adapts better to the circadian phase shift when travelling westward. Therefore, a first maximum limit for the clockwise shifting may be greater than a second maximum limit for the counter-clockwise shifting. For example, the first maximum limit may be one hour per day while the second maximum limit may be between 0.5 to 0.7 hours per day.

In yet another exemplary embodiment, the maximum limit(s) may be a function of direction of the change of the sleep gate with respect to an optimal sleep gate for a local sunlight rhythm (proportional to light exposure) of the user's location. Different geographical locations on Earth have different sunlight rhythms and the sunlight rhythm affects the natural circadian rhythm for the particular location. Furthermore, the sunlight rhythm at the location of the user affects the adaptation rate of the natural sleep gate, and this embodiment quantizes this characteristic, thereby providing a more accurate estimate of the circadian phase adaptation rate. In this embodiment, a target sleep gate timing may be determined on the basis of the local sunlight rhythm of the user's location. If the change to the timing of the sleep gate determined from the measured circadian phase is towards the target sleep gate timing, the maximum limit(s) may follow the principles described in the preceding paragraph. However, if the change to the timing of the sleep gate determined from the measured circadian phase is away from the target sleep gate timing, the maximum limit(s) may be reduced. In such a case, the difference between the first and second maximum limit may also be negated, meaning that the same maximum limit is used in both when the sleep gate shifts clockwise and when it shifts counter-clockwise. The maximum limit in such a case may be smaller than 0.5 hours per day or smaller than 0.4 hours per day.

FIGS. 5a and 5b illustrate non-limiting exemplary display views of sleep gate timing and accuracy.

Referring to FIG. 5a, the daily schedule 501 of the user is illustrated on the display of the user device 102 as blocks of hours. The first hours of the sleep window 502 are illustrated as dotted blocks. The sleep gate 503 is illustrated as a ruled patch placed in the beginning of the sleep window 502. The size (height) of the patch indicates the time period of the sleep gate 503. The reliability of the sleep window is indicated by the thickness of the ruling of the patch.

Referring to FIG. 5b, the sleep gate 504 is illustrated with a larger patch compared to the sleep gate 503 illustrated in FIG. 5a. This indicates that the time period of the sleep gate 504 is longer. The thickness of the ruling the patch is smaller indicating that the reliability of the sleep gate 504 is poorer compared to the reliability of the sleep gate 503.

As said above, the embodiment illustrated in FIGS. 5a and 5b is just an example of indicating the sleep gate timing and reliability, and numerous other display techniques may be utilised. The reliability metric value may be provided in the form of a score value within a determined range, e.g. 1 to 10, 0 to 10, or 0 to 100. The reliability metric value may be provided in a verbal form, e.g. “High”, “Compromised”, and “Poor”. The reliability metric value may be provided in a coloured form of the sleep gate. For example, the sleep gate may be indicated in the form of a time interval (numeral or graphic), and the colour of the time interval may indicate the reliability, e.g. green for high reliability, orange or yellow for compromised reliability, and red for poor reliability.

The blocks and related functions described above in FIGS. 2 to 4 are in no absolute chronological order, and some of the blocks may be performed simultaneously or in an order differing from the given one. Other functions can also be executed between the blocks or within the blocks. Some of the blocks or part of the blocks can also be left out or replaced by a corresponding block or part of a block, for example, determining the sleep gate, determining the reliability metric value, or causing displaying to the user can be left out or replaced with each other.

The techniques described herein may be implemented by various means so that an apparatus implementing one or more functions/operations described above with an embodiment/example, for example by means of any of FIGS. 1 to 5 and any combination thereof, comprises not only prior art means, but also means for implementing the one or more functions/operations of a corresponding functionality described with an embodiment, for example by means of any of FIGS. 1 to 5 and any combination thereof, and it may comprise separate means for each separate function/operation, or means may be configured to perform two or more functions/operations. For example, one or more of the means for one or more functions/operations described above may be software and/or software-hardware and/or hardware and/or firmware components (recorded indelibly on a medium such as read-only-memory or embodied in hard-wired computer circuitry) or combinations thereof. Software codes may be stored in any suitable, processor/computer-readable data storage medium(s) or memory unit(s) or article(s) of manufacture and executed by one or more processors/computers, hardware (one or more apparatuses), firmware (one or more apparatuses), software (one or more modules), or combinations thereof. For a firmware or a software, implementation can be through modules (for example procedures, functions, and so on) that perform the functions described herein.

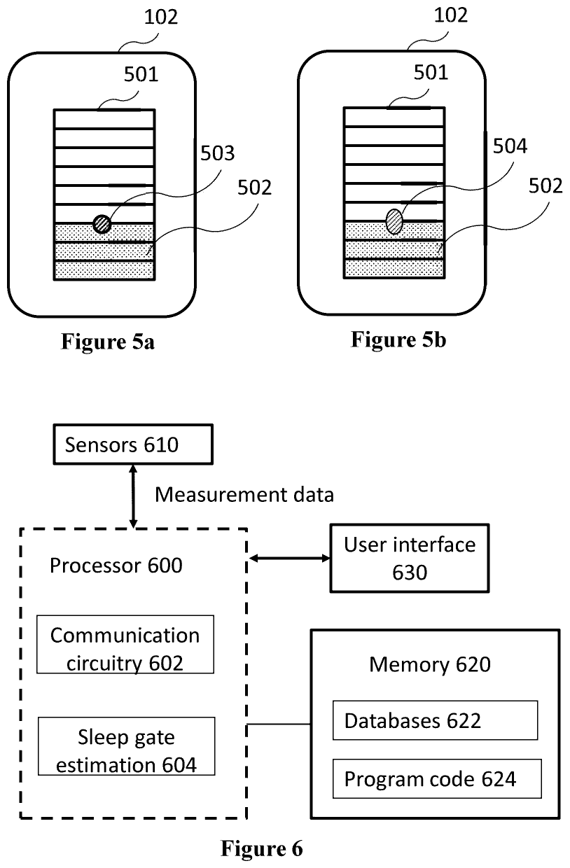

FIG. 6 is a simplified block diagram illustrating a structure of an apparatus (device, equipment) 600 according to an embodiment and configured to perform at least some functionality described above for estimating sleep gate, for example by means of FIGS. 1 to 5 and any combination thereof. The apparatus may be applicable to or comprised in the user device. In other embodiments, the apparatus is applicable to or comprised in a sensor device, a wearable device, or a server computer. The apparatus may comprise at least one processor 600 or processing circuitry and at least one memory 620 including a computer program code 624, wherein the at least one memory 620 and the computer program code 624 are configured, with the at least one processor 600, to cause the apparatus to carry out the functions described above in connection with the processing circuitry. The processor 600 may comprise a communication circuitry 602 as a sub-circuitry configured to handle wireless connection with one or more sensor devices 610 or internal connection between computer program modules through one or more application programming interfaces (APIs) in the apparatus. The sensor device(s) 610 may be comprised in the apparatus, be external to the apparatus, or comprise both internal and external sensor devices. The sensor device(s) 610 may comprise at least one of the following sensors: a heart activity sensor measuring the ECG, BCG, or PPG, a motion sensor or an inertial sensor measuring motion, an EEG sensor measuring the EEG, an EOG sensor measuring the EOG, a bioimpedance sensor measuring the bioimpedance or another galvanic property from a skin, and a respiratory rate sensor measuring the respiratory rate. The communication circuitry 602 may be configured to receive measurement data form the sensor device(s) 610. The communication circuitry 602 may be configured to output sleep gate estimation and/or other information through an API as described above.

The processor may comprise a sleep gate estimation module 604 configured to determine the sleep time and the reliability metric values according to any one of the embodiments of FIGS. 2 to 4 and any combination thereof. The sleep gate estimation module 604 may be configured by the computer program code 624 to map the obtained measurement data to the sleep gate and the reliability metric values. The memory 620 may store a database 622 that provides rules for mapping of the obtained measurement data to the sleep gate estimation. When executing the processes according to any one of the above-described embodiment, the sleep gate estimation module 604 may output the sleep gate guidance to the user via one or more interface (IF) entities 630, such as one or more user interfaces comprised in the apparatus or being external to the apparatus. The one or more interface entities 630 are entities for receiving and transmitting information, such as communication interfaces comprising hardware and/or software for realising communication connectivity according to one or more communication protocols, or for realising data storing and fetching, or for providing user interaction via one or more user interfaces. The user interface may comprise a display screen or a display module for displaying the sleep gate guidance. The user interface may also comprise an input device for inputting information such as the target sleep or sleep occurrence.

As used in this application, the term “circuitry” refers to all of the following: (a) hardware-only circuit implementations such as implementations in only analog and/or digital circuitry; (b) combinations of circuits and software and/or firmware, such as (as applicable): (i) a combination of processor(s) or processor cores; or (ii) portions of processor(s)/software including digital signal processor(s), software, and at least one memory that work together to cause an apparatus to perform specific functions; and (c) circuits, such as microprocessor(s) or a portion of a microprocessor(s), that require software or firmware for operation, even if the software or firmware is not physically present.

This definition of “circuitry” applies to all uses of this term in this application. As a further example, as used in this application, the term “circuitry” would also cover an implementation of merely a processor (or multiple processors) or portion of a processor, e.g. one core of a multicore processor, and its (or their) accompanying software and/or firmware. The term “circuitry” would also cover, for example and if applicable to the particular element, a baseband integrated circuit, an applicationspecific integrated circuit (ASIC), and/or filedprogrammable grid array (FPGA) circuit for the apparatus according to an embodiment.

The processes or methods described in FIGS. 2 to 4 and any combination thereof may also be carried out in the form of a computer process defined by a computer program. The computer program may be in source code form, object code form, or in some intermediate form, and it may be stored in some sort of carrier, which may be any entity or device capable of carrying the program. Such carriers include transitory and/or nontransitory computer media, e.g. a record medium, computer memory, readonly memory, electrical carrier signal, telecommunications signal, and software distribution package. Depending on the processing power needed, the computer program may be executed in a single electronic digital processing unit, or it may be distributed amongst a number of processing units.

It will be obvious to a person skilled in the art that, as the technology advances, the inventive concept can be implemented in various ways. The invention and its embodiments are not limited to the examples described above but may vary within the scope of the claims.

What is claimed is:

1. A computerimplemented method comprising:
obtaining measurement data measured, from a user, by at least one sensor device;
determining, using the measurement data obtained, a circadian rhythm of the user, wherein the circadian rhythm comprises a circadian phase;
determining, using the measurement data obtained, a sleepwake rhythm of the user;
determining, using the circadian phase determined, a sleep gate, wherein the sleep gate indicates a time window for falling asleep suitable for the circadian rhythm of the user;
determining, using at least the sleepwake rhythm determined, a reliability metric value indicating a timing accuracy of the sleep gate; and
causing displaying, to the user via a user interface, the sleep gate and the reliability metric value.

2. The computerimplemented method according to claim 1, further comprising:
determining a deviation between the circadian rhythm and the sleepwake rhythm; and
determining, using the deviation determined, the reliability metric value, wherein a greater deviation results in a reliability metric value indicating a poorer timing accuracy.

3. The computerimplemented method according to claim 1, wherein the determining the reliability metric value comprises selecting the reliability metric value amongst at least a first reliability metric value and a second reliability metric value, wherein the first metric value indicates a higher timing accuracy than the second metric value.

4. The computerimplemented method according to claim 3, further comprising:
detecting a latest sleep event of the user;
determining a time interval elapsed after the latest sleep event detected;
determining whether the latest sleep event is a primary sleep event or a secondary sleep event;
selecting, if the latest sleep event detected is a primary sleep event or if the latest sleep event detected is a secondary sleep event and if the time interval elapsed is above a predetermined time interval threshold, the first reliability metric value as the reliability metric value; and
selecting, if the latest sleep event detected is a secondary sleep event and if the time interval elapsed is below the predetermined time interval threshold, the second reliability metric value as the reliability metric value.

5. The computerimplemented method according to claim 4, wherein the time interval elapsed comprises a time interval from the latest sleep event to the beginning of the sleep gate.

6. The computerimplemented method according to claim 3, further comprising:
detecting a secondary sleep event of the user:
determining a sleep time of the secondary sleep event; and
selecting, if the sleep time of the secondary sleep event is below a predetermined sleep time threshold, the first reliability metric value as the reliability metric value;
selecting, if the sleep time of the secondary sleep event is above the predetermined sleep time threshold, the second reliability metric value as the reliability metric value.

7. The computerimplemented method according to claims 3, further comprising:
estimating, using the sleepwake rhythm determined and a target sleep time preset for the user, a sleep debt value;
selecting, if the sleep debt value is below a predetermined sleep debt threshold, the first reliability metric value as the reliability metric value; and
selecting, if the sleep debt value is above the predetermined sleep debt threshold, the second reliability metric value as the reliability metric value.

8. The computerimplemented method according to claims 3, further comprising:
storing, in a memory, a sleep history of the user, wherein the sleep history is based on the measurement data measured during one or more earlier primary sleep events of the user;
detecting a lack of measurement data indicating a primary sleep event of the user within a preceding 24hour time period;
generating, in response to the said detecting, an artificial measurement data of an artificial sleep event using the sleep history stored; and
selecting, in response to the said detecting, a third reliability metric value as the reliability metric value, wherein the third reliability metric value indicates a lower timing accuracy than the first reliability metric value.

9. The computerimplemented method according to claims 3, further comprising:
determining, in response to not being able to determine the circadian rhythm, the sleep gate of a preceding day as the sleep gate; and
selecting, in response to the determining the sleep gate of the preceding day as the sleep gate, a fourth reliability metric value as the reliability metric value, wherein the fourth reliability metric value indicates a lower timing accuracy than the first reliability metric value.

10. The computerimplemented method according to claim 1, further comprising:
storing, in a memory, a maximum limit defining how much at most a timing of the sleep gate can be changed at a time;
determining, using the circadian phase detected, that the circadian phase has shifted;
determining, in response to determining that the circadian phase has shifted, a new timing for the sleep gate, wherein the new timing is limited by the maximum limit.

11. The computerimplemented method according to any of the claim 1, wherein the measurement data obtained comprises body temperature data of the user, further comprising:
determining, using the body temperature data, the circadian rhythm; and
determining, using the circadian rhythm and the body temperature data, the sleep gate.

12. An apparatus comprising:
at least one processor; and
at least one memory including computer program code, the at least one memory and computer program code being configured to, with the at least one processor, cause the apparatus to perform a method comprising:
obtaining measurement data measured, from a user, by at least one sensor device;
determining, using the measurement data obtained, a circadian rhythm of the user, wherein the circadian rhythm comprises a circadian phase;
determining, using the measurement data obtained, a sleepwake rhythm of the user;
determining, using the circadian phase determined, a sleep gate, wherein the sleep gate indicates a time window for falling asleep suitable for the circadian rhythm of the user;
determining, using at least the sleepwake rhythm determined, a reliability metric value indicating a timing accuracy of the sleep gate; and
causing displaying, to the user via a user interface, the sleep gate and the reliability metric value.

13. A computer program product comprising a program code that, when executed by a computer, causes execution of a method comprising:
obtaining measurement data measured, from a user, by at least one sensor device;
determining, using the measurement data obtained, a circadian rhythm of the user, wherein the circadian rhythm comprises a circadian phase;
determining, using the measurement data obtained, a sleepwake rhythm of the user;
determining, using the circadian phase determined, a sleep gate, wherein the sleep gate indicates a time window for falling asleep suitable for the circadian rhythm of the user;
determining, using at least the sleepwake rhythm determined, a reliability metric value indicating a timing accuracy of the sleep gate; and
causing displaying, to the user via a user interface, the sleep gate and the reliability metric value.

14. The computer program product of claim 13, wherein the computer program product is comprises a nontransitory computerreadable medium.

15. The apparatus of claim 12, further comprising a computer system comprising means for carrying out at least the method.

16. The computer program product of claim 13, further comprising a computerreadable program code that, when executed by a computer system, causes execution of the method.
